(19) United States
(12) Patent Application Publication
(10) Pub. No.: US 2011/0082558 A1
(43) Pub. Date: Apr. 7, 2011

(54) ARTIFICIAL KNEE JOINT

(76) Inventors: Sung-kon Kim, Ansan (KR); Krotha Srinivasareddy, Ansan (KR)

(21) Appl. No.: 12/898,989

(22) Filed: Oct. 6, 2010

(30) Foreign Application Priority Data
Oct. 7, 2009 (GB) GB 0917489.7

(51) Int. Cl. A61F 2/38

(52) U.S. Cl. 623/20.31

(57) ABSTRACT

An artificial knee joint comprise a femoral component and tibial component. The posterior side of the femoral component comprises medial and lateral condyles, wherein the width and offset of the posteromedial condyle is greater than the width and offset of the posterolateral condyle. At the posterior the tibial bearing component comprises medial and lateral articulating surface geometries, wherein the posterior slope of the lateral articulating geometry is greater than the posterior slope of the medial articulating geometry. The medial articulating surface geometry of the tibial bearing component supports the medial condyle of the femoral component and the lateral articulating surface geometry of the tibial bearing component supports the lateral condyle of the femoral component. The greater slope of the lateral articulating geometry allows the femoral component condyle to roll down to the posterior during knee flexion. This invention of an artificial knee joint for a prosthetic knee implant system facilitates deep knee flexes beyond 130 degrees.

### FIELD OF THE INVENTION

The present invention concerns medical prosthetic devices. More specifically, this invention relates to a prosthetic knee implant system and to an artificial knee joint for a prosthetic knee implant system that allows the knee to deep flex. The invention, furthermore, relates to a femoral component and tibial component orthopaedic knee implant for use in conjunction with a total knee arthroplasty (TKA), wherein the femoral component condyles accommodate deep flexion and minimize the impingement of the femur with the tibial component.

### BACKGROUND TO THE INVENTION

Over the last three decades total knee replacement (TKR) surgery has evolved into a reproducibly successful procedure benefiting hundreds of thousands of patients each year. Greater understanding of proper implant design and standardization of surgical technique has occurred. And as the procedure has matured over the last decade, the pace of its evolution has slowed. However, two recent developments have quickened that pace of change.

The first of these is the development of more minimally invasive methods of performing TKR. Minimally invasive surgery (MIS) has benefited patients by diminishing immediate postoperative pain, shortening hospital stay and rehabilitation time, and overall making the TKR surgical experience easier to endure. To a large extent it has changed forever the way in which TKR surgery is performed and with it patients' expectations.

The second of these developments has been the concept of providing for patients a prosthetic knee that allows for deeper flexion. TKR patients who have successfully achieved deep flexion can lead a more normal, easier and freer lifestyle having to make fewer minute to minute and day to day concessions to their artificial knee. Simple lifestyle events, such as putting on shoes and socks, foot hygiene, sitting in movie theatres and airplane seats, getting into and out of the back seat of cars, and getting into low spots around the home, all are made simpler and more comfortable when deep flexion in a TKR has been achieved.

The knee is the biggest, most complicated and most incongruent joint in the human body. Because the knee is located between the body's two longest lever-arms, it sustains high forces. Over the past 25 years, total knee replacement (TKR) has been one of the most successful operating procedures developed. In more than 95% of TKR cases, TKR has been shown to restore the ability to walk without limp or pain. Historically, TKR has been used primarily on older patients with osteoarthritis. However, because of the amazing success of TKRs, they are now being considered in younger, more active patients. This presents an entirely different design challenge. Because the patients are both younger and more active, durability and performance become much more important factors. Durability in TKRs consists mainly of wear and loosening. Performance consists mainly of range of motion, stability, and gait analysis (stair climbing ability).

The knee functions to allow movement of the leg and is critical to normal walking. The knee flexes normally to a maximum of 135 degrees and extends to 0 degrees. The bursae, or fluid-filled sacs, serve as gliding surfaces for the tendons to reduce the force of friction as these tendons move. The knee is a weight-bearing joint. Each meniscus serves to evenly load the surface during weight-bearing and also aids in disbursing joint fluid for joint lubrication. Many factors are presently being experimented with to try to reduce wear. One approach is by improving the quality of the condyle of femoral component and quality of the polyethylene component. As more polyethylene is cross-linked, its wear properties increase and the strength decrease. Studies are being done to find the optimal amount of cross-linking as well as the best sterilization technique.

Most current knee prostheses employ designs which strive to simulate the articulation of the natural knee by attempting to duplicate the geometry of the articular surface of the natural knee. Many of these knee prostheses have been found to experience relatively high stresses placed upon the tibial bearing member as a result of loads encountered during articulation of the knee prosthesis and difficulties in balancing the tension in the collateral ligaments of the knee for optimum performance. Such high stresses and imbalances in the tension in the collateral ligaments have an adverse effect on performance and reliability, and usually lead to a limited service life.

Deep flexion TKR is arbitrarily defined as a knee that achieves flexion greater than 115 to 130 degrees. In the early development of the resurfacing TKR surgery, 90 degrees of flexion was considered sufficient or even ideal. Indeed, there was concern that with deeper flexion posterior instability would be risked and that polyethylene wear would be enhanced. Concerns regarding these issues still exist. These concerns are valid if the surgeon achieves deep TKR flexion without adhering to strict surgical principles regarding stability and proper tibiofemoral tracking. To achieve a high flexion TKR that is symptom free and stable requires a thorough understanding of normal knee kinematics including the concept of femoral rollback and the need for physiologic posterior stability. The goal of deep flexion TKR surgery is to obtain deep flexion while maintaining a balanced, kinematically functional, stable knee.

Most TKRs, however, include femoral components that are designed to accommodate knee joint articulation from a position of slight hyper extension to approximately 115 degrees to 130 degrees of flexion. However, the healthy human knee is capable of a range of motion (ROM) approaching 170 degrees of flexion, and a ROM of around 155 degrees is required for deep kneeling and squatting as may be required during some sporting, religious or cultural events. Thus there is a need for an improved TKR femoral component that accommodates knee flexion, under optimal conditions, of more than 130 degrees (high flexion).

As the normal knee flexes, femoral rollback occurs. The lateral femoral condyle, having a larger radius of curvature, rolls back farther posterior than the medial femoral condyle. This rollback is guided by the posterior cruciate ligament (PCL). The asymmetric rollback results in the tibia internally rotating relative to the femur during flexion.

In the TKR patient, normal kinematics must also be guided by a functioning PCL. If the TKR is posteriorly unstable, paradoxical anterior slide of the femur on the tibia occurs and normal knee kinematics is not exhibited. This paradoxical anterior slide of the femur on the tibia during flexion can be a cause of undesirable symptoms. These may include difficulty with stairs and inclines (particularly going down), soreness when the knee is flexed and loaded, such as with recreational athletic activities, and paradoxical anterior femoral slide on the tibia can be a cause of intermittent effusions as the femur repetitively stresses and irritates the anterior capsule of the knee. In addition, anterior sliding of the femur can cause earlier impingement of the posterior polyethylene on the back of the femur, thus preventing high flexion from occurring. To achieve a high-flexion, symptom-free knee, normal kinematics must be understood. It is not satisfactory to achieve deep flexion knee arthroplasty if it is posteriorly unstable and functionally symptomatic due to altered knee kinematics.

During flexion the lateral femoral condyle moves posteriorly while the position of the medial femoral condyle is relatively stationary. This produces relative internal tibial rotation or external femoral rotation during knee flexion. At high degrees of flexion the lateral femoral condyle may displace posteriorly to the point that it is partially subluxed (dislocated) from the tibial surface. The combined lateral femoral roll-back and femoral external rotation are essential to permit large degrees of knee flexion.

The kinematic behaviour of the knee at 150° was markedly different from that measured at other flexion angles. Muscle loads appear to play a minimal role in influencing tibial translation and rotation at maximal flexion. The results imply that the knee is highly constrained at high flexion. This stability may be due in part to the effects of soft tissues. Posterior capsule and menisci may contact into the concave surface of the femoral condyles. The stability of the knee at high flexion will thus be at the expense of high stresses in the surrounding soft tissues. Therefore, patients with meniscal or capsular repair should avoid high flexion during their early healing phase. The present invention establishes that knee arthroplasty designs, particularly femoral components for high flexion, need design modifications that will provide strong posterior stability while allowing sufficient femoral rollback at high flexion angles. The present invention aims to address this need.

In the posterior cruciate retaining knee, the tibial bearing implant polyethylene design should allow for anatomic rollback guided by the posterior cruciate ligament. However, an excessively flat polyethylene design risks peak point contact stresses and posterior edge loading (if rollback is excessive) resulting in increased polyethylene wear. Thus, some congruence is required. In addition, due to the concerns regarding posterior instability in deep flexion, multiple polyethylene constraint options enhancing stability are necessary. Posteriorly lipped or dished implants, anteriorly lipped implants that are effective at enhancing stability when descending stairs, and various levels of PCL substituting implants should be available. “Anterior lipped” polyethylene inserts can theoretically be particularly effective in helping to prevent paradoxical anterior sliding of the femur on the tibia in flexion.

The present invention aims to provide a prosthetic device with relatively normal kinematic function after total knee arthroplasty, allowing high flexion beyond 130 degrees while minimizing impingement with the tibial base plate when the knee flexes.

Prior art U.S. Pat. No. 5,549,688 and U.S. Pat. No. 7,264,635 disclose prosthetic femoral parts that have some adaptation to suit high flexure but are in fact far from ideal for that purpose. In U.S. Pat. No. 5,549,688, the medial condyle is offset more than the lateral condyle from the posterior to the anterior side and in U.S. Pat. No. 7,264,635, the posterolateral condyle is offset more than the posteromedial condyle. For U.S. Pat. No. 5,549,688, the way the femoral prosthetic features are configured leads to the knee failing to maintain adequate tibiofemoral contact during high flexion and fails to provide optimal clearance for the patellar tendon. This design actually fails in kinematics of the knee to continue into deep flexion angles. As for U.S. Pat. No. 7,264,635, the configuration of the femoral prosthetic features leads to increase in the tightness of the lateral retinacular ligament during high flexion and the knee fails in kinematics to continue into deep flexion angles.

The present invention of artificial knee joint aims to provide a femoral component for achieving the desired high flexion while minimizing the impingement with the tibial base plate.

A prosthetic tibial bearing part of relevance is known from U.S. Pat. No. 7,060,101. In this art the lateral bearing surface is higher than the medial bearing surface in the posterior side. In this way the lateral ligament is tightened progressively more than the medial ligament, resulting in increased stability in the lateral compartment. However, the normal kinematic function after total knee arthroplasty fails to produce greater posterior translation of lateral than the medial femoral condyle during knee flexion. Due to this condition the knee fails to achieve deep flexion beyond 130 degrees and fails in proper flexion gap after total knee arthroplasty.

It is an object of the present invention to minimize the above-discussed problems of the prior art.

### SUMMARY OF THE INVENTION

According to the present invention there is provided an artificial knee joint which comprises: a femoral component to be attached to a femur, the femoral component having a posteromedial condyle and a posterolateral condyle; and a tibial component to be attached to a tibia, the tibial component having a medial articulating geometry and a lateral articulating geometry, wherein in the femoral component the width and offset of the posteromedial condyle is greater than the width and offset of the posterolateral condyle and in the tibial component the angle of the posterior slope of the lateral articulating geometry is greater than the angle of the posterior slope of the medial articulating geometry.

The present artificial knee joint invention thus includes both femoral component design and tibial bearing component design so that the knee prosthesis as a whole may have the desired kinematic behaviour for deep flexion.

In the femoral part of the knee prosthetic device the focus is on the condyles design for achieving maximum flexion at the knee, for bends beyond 130 degrees. In this present invention the femoral component is designed in such a way that the width and offset of the posteromedial condyle is greater than the width and offset of the posterolateral condyle.

Posterior offset is the distance from the posterior femoral condyle to the femoral cortex. In the present invention the femoral component's posteromedial condyle is designed more offset than the femoral component's posterolateral condyle. With the larger posterior offset of the medial side greater knee flexion can be achieved before posterior impingement occurs. Considering the knee kinematics at high degrees of flexion the position of the medial condyle on the tibial plateau is relatively constant, similar to a ball and socket mechanism, but at high degrees of flexion, the lateral condyle nearly subluxes off the posterior tibial plateau. On the lateral side posterior impingement between the femur and tibia does not generally occur as compared to the medial side.

According to the knee kinematics at high degree of flexion, in the present invention the femoral component has the posteromedial condyle offset more than the posterolateral condyle. This helps to minimize the impingement that occurs on the posteromedial side due to the medial condyle position at high degrees of flexion. In the present invention the femoral component has a posteromedial condyle width that is more than the width of the posterolateral condyle. As mentioned previously, femoral rollback occurs when the knee flexes and the lateral femoral condyle, having a larger radius of curvature, rolls back further posterior than the medial femoral condyle. During knee flexion the position of the medial condyle on the tibial plateau is relatively constant. At this moment the posteromedial condyle requires more contact area with the tibial articulating surface to avoid the unnecessary constraints due to contact stresses.

The present invention mitigates against this problem by providing a wider posteromedial condyle than posterolateral condyle. As a result, contact stresses are distributed over a wide area, generally with slightly dished curvature, thereby minimising or avoiding unnecessary constraint. An advantage of the present invention is that this configuration allows more posterior offset on the posterior condyle and with more posterior offset, greater knee flexion can be achieved before posterior impingement occurs. Another advantage of the present invention's wider posteromedial condyle than posterolateral condyle is that this minimizes the contact stress with the tibial articulating surface at the medial side, when the knee flexes beyond 130 degrees.

The second design criterion of the present invention concerns the tibial bearing geometry. According to the invention the tibial bearing component of knee prosthetic has asymmetric medial and lateral articulating surfaces at the posterior side. This contributes to the prosthetic allowing for maximum flexion as the knee bends beyond 130 degrees. In this present invention in the tibial bearing component the posterior slope of the lateral articulating geometry is greater than the posterior slope of the medial articulating geometry. Stated another way, the posterior slope of the lateral articulating geometry drops by a greater amount and to a lower level at the rear edge of the tibial component (200) than does the posterior slope of the medial articulating geometry (202).

The greater slope of the lateral plateau allows the femoral condyle to roll down the posterior lateral slope during knee flexion. Due to this arrangement we can achieve the goal of relatively normal kinematic function after total knee arthroplasty at high degrees of flexion.

The present invention allows for relatively normal kinematic function after total knee arthroplasty, in part as result of the tibial bearing component being configured with asymmetric articulating geometries, lateral and medial, on the posterior side as described. The greater posterior slope of the lateral articulating surface than the medial allows greater posterior translation of the lateral condyle of the femoral component. This movement simulates normal kinematic function after total knee arthroplasty. Furthermore the medial articulating surface that has less posterior slope than the lateral articulating surface effectively has a posterior lip. This configuration prevents medial condyle translation towards the posterior and further aids the prosthesis in better imitating normal kinematic function after total knee arthroplasty.

A third design criterion of the present invention is to provide for a more perfect congruence between femoral condyles and tibia bearing surfaces in the frontal plane. As for the analysis result, a substantially perfect congruence is obtained when the concave surfaces of the tibia insert has a curvature radius that is close to equal to the curvature radius of the femoral condyle radius in flexion, i.e. beyond 80 degrees or 90 degrees in the frontal plane, thereby maximizing contact area and reducing stress that can lead to premature polyethylene wear. A ratio of 1.07:1 is found to be well suited to achieve the desired results.

The present invention also provides a knee prosthetic including a femoral component such as previously defined and a tibia insert laid onto a tibia plate in the sagittal plane, with the tibia insert including an upper concave surface which co-operates with the external surface of the condyles, the curvature radius of the external surface of the insert being substantially equal to the curvature radius of the femoral condyle radius.

### BRIEF DESCRIPTION OF THE DRAWINGS

A preferred embodiment of the present invention will now be more particularly described, by way of example only, with reference to the accompanying drawings, wherein:

FIG. 1 is an exploded, schematic view of a total knee joint prosthetic system embodying the present invention, in association with a proximal portion of a tibia and a distal portion of a femur;

FIG. 2 is a 3D isometric view of a femoral component part of the system;

FIG. 3 is a posterior view of the femoral component;

FIG. 4 is a side view of the femoral component and shows the difference in medial and lateral condyle offset at the posterior;

FIG. 5 is a side view of the posterior condyle when the knee is in high flexion;

FIG. 6 is a sagittal view of the lateral and medial condyles articulating with the lateral and medial tibial plateau;

FIG. 7 is an isometric view of the tibial bearing component of the knee prosthetic;

FIG. 8 is a posterior view of the tibial bearing component and shows the difference in medial and lateral articulating geometries

FIG. 9 is a sagittal plane view of the medial and lateral articulating geometries of the tibial bearing component and shows the difference in posterior slope of the medial and lateral articulating geometries;

FIG. 10 is a sagittal plane view of the combined medial and lateral articulating geometries of the tibial bearing component; and

FIG. 11 is a frontal view cross-section of the femoral part and tibia insert of the preferred embodiment.

Corresponding reference characters indicate corresponding parts throughout the several views.

### DESCRIPTION OF THE PREFERRED EMBODIMENT

As used herein, the following directional definitions apply. Anterior and posterior mean nearer the front or nearer the back of the body respectively. Thus, for the knee joint described herein, anterior refers to regions of the knee that are nearer the front of the body when the leg is in an extended position. Proximal and distal mean nearer to or further from the root of the structure, respectively. For example, the distal femur is the region of the femur that is at or nearer the knee joint while the proximal femur is at or nearer to the hip joint. Finally, the adjectives medial and lateral mean nearer the sagittal plane or further from the sagittal plane respectfully. The sagittal plane is an imaginary vertical plane through the middle of the body that divides the body into right and left halves.

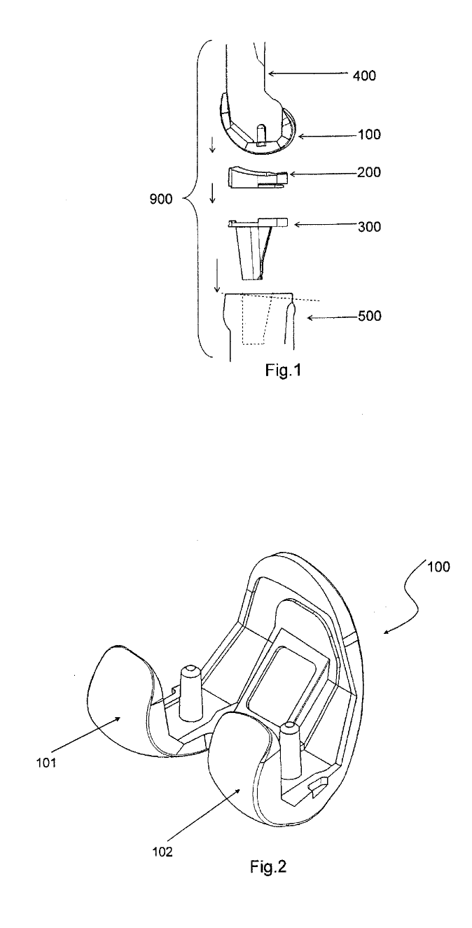

Referring now to FIG. 1, this is an exploded view of the total knee joint 900, which includes femoral component 100, tibial component (a.k.a. tibial insert) 200, tibial base component 300, femur bone 400 and tibia bone 500. The key features are in the design of the femoral component 100 and tibial (insert) component 200.

Referring now to FIG. 2, this is an isometric view of the femoral component 100 of FIG. 1. On the posterior side the femoral component 100 comprises medial 102 and lateral 101 condyles, wherein the width and offset of the posteromedial condyle 102 is greater than the width and offset of the posterolateral condyle 101. The medial condyle is more offset than the lateral condyle in the posterior side and from the distal portion to the anterior side the lateral condyle is more offset than the medial condyle as shown in the FIG. 4, where H and K are the condyle offsets on lateral and medial condyles of the femoral component, respectively.

In the present invention the posterior condyle design of the femoral component is the main focus point for achieving maximum range of motion when the knee flexes. The femoral component 100 may comprise any biocompatible material having the mechanical properties necessary to function as a human knee distal femoral prosthesis. Preferably the femoral component 100 comprises titanium, titanium alloy, cobalt chrome alloy, stainless steel, or a ceramic.

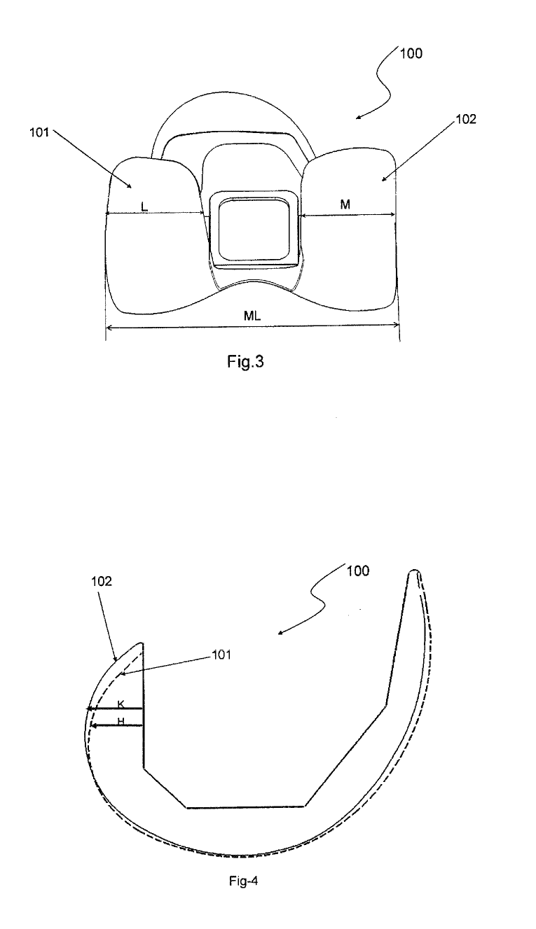

Referring now to FIG. 3, ML is the mediolateral distance width, and this means for both femoral and tibial components, the maximum width of the components in the frontal elevation. Here M is the width of the posterior medial condyle 102 and L is the width of the posterior lateral condyle 101.

The femoral condyles are designed to have the width of the posterior medial condyle 102 greater than the width of the posterior lateral condyle 101.

Considering the kinematics of the replaced knee, fluoroscopic studies have consistently demonstrated paradoxical motion in which the femur moves anteriorly during flexion and posteriorly during extension. As the knee flexes, the femur moves paradoxically from a posterior to an anterior position on the tibia. With the knee in high flexion, flexion is limited by posterior bony or soft tissue impingement (arrow) as shown in FIG. 6. With the knee in high flexion the maximum range of motion is achieved by minimizing or avoiding such impingement. To avoid such impingement is a primary objective of the present invention.

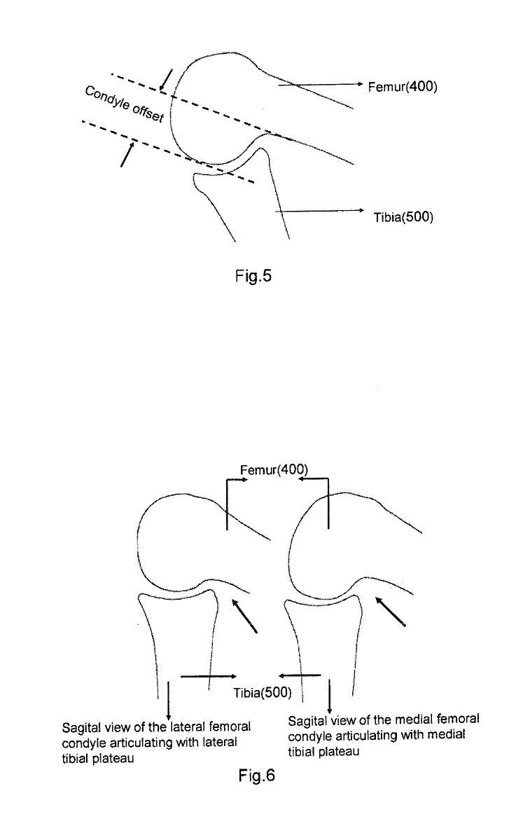

Posterior offset is the distance from posterior femoral condyle to femoral cortex (between arrows) as shown in FIG. 5. With larger posterior offset, greater knee flexion can be achieved before impingement as shown in FIG. 6. In the present invention larger posterior offset is the main design consideration.

In the cited prior art referred to earlier a larger posterior offset of the femoral component is proposed but in the present invention we have established that offset variations between the posterior medial and lateral condyles are neccessary. Considering the kinematics of the knee at high flexion due to external rotation the position of the medial condyle on the tibial plateau is relatively constant, similar to a ball-and socket mechanism and the lateral condyle translates posteriorly. Impingement between the femur and the tibia at maximum flexion occurs on the medial side. We have designed the medial condyle to be larger than the lateral condyle so that the posterior offset of the medial condyle is greater than that of the lateral condyle as shown in the FIG. 4.

Referring now to FIG. 6, this shows the lateral femoral condyle articulating with the lateral tibial plateau (left image) and the medial femoral condyle articulating with the medial tibial plateau (right image) when the knee is in high flexion. Since the medial femoral condyle is larger than the lateral condyle, posterior offset is greater medially than laterally. If there is no posterior movement of the femur on the tibia during knee flexion, posterior impingement occurs between the lateral femoral cortex and tibial plateau, limiting knee flexion (arrow) as shown. As afore-mentioned, when the knee is in high flexion the lateral femoral condyle translates posteriorly. The lateral femoral condyle moves posteriorly relative to the tibia so that posterior impingement does not occur. As a result of posterior movement of the lateral femoral condyle, flexion is increased before posterior impingement occurs.

Our femoral component has more posterior medial condyle offset than posterior lateral condyle offset and we have found that 1 to 3 mm more offset on the medial condyle gives good results on high flexion. The preferred optimal offset of the medial condyle is 2.5 mm more than the lateral condyle offset. Larger medial posterior offset allows for greater flexion and the widened femoral condyle allows for increased contact area in deep flexion.

With the knee in high flexion, lowered height of the lateral condyle at the posterior side decreases the tightness of the lateral retinacular ligament. This is a significant advantage of the present invention over the prior art.

The width of the medial condyle is more than that of the lateral condyle at the posterior side as shown in the FIG. 3. As mentioned above, when the knee is in high flexion the position of the medial condyle on the tibial plateau is relatively constant and the lateral condyle translates posteriorly. At high flexion the medial condyle needs more contact area on the tibial plateau for better stability and to avoid unnecessary constraint. Since our medial condyle width is more than lateral condyle width the contact stress is distributed over a wide area and avoids unnecessary constraint. We have found that 1 to 2 mm more width on the medial condyle gives good results in minimizing the contact stress on the medial side. Optimally the width on the medial condyle is 1.5 mm more than the width on the lateral condyle.

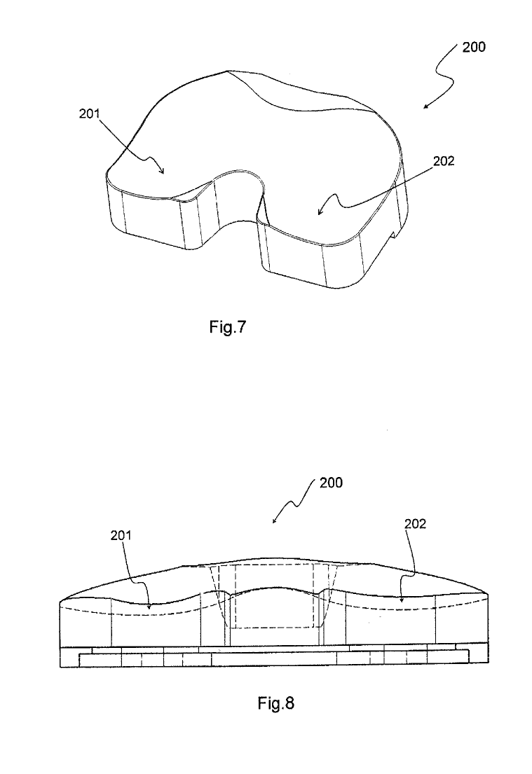

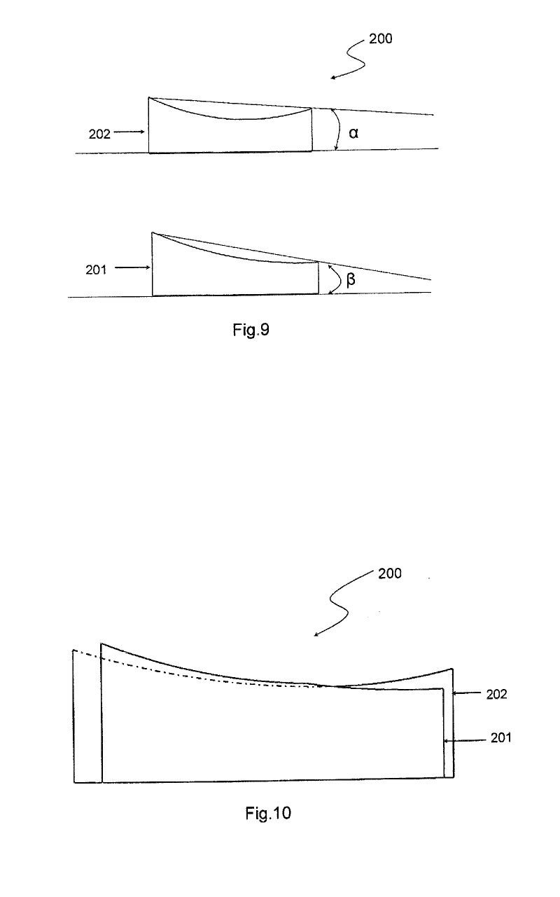

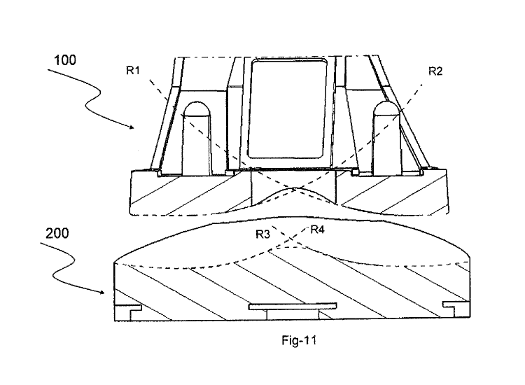

Referring now to FIG. 7 and FIG. 8, these show the tibial bearing component 200 of the total knee replacement system. The bearing geometry of the component 200 comprises a medial articulating surface 202 and a lateral articulating surface 201 as shown in FIG. 8. In the tibial bearing component 200 the posterior slope of the lateral articulating geometry 201 is greater than the posterior slope of the medial articulating geometry 202, as shown in FIG. 9 and FIG. 10.

As the normal knee flexes, femoral rollback occurs. The lateral femoral condyle, having a larger radius of curvature, rolls back further posterior than the medial femoral condyle on the tibial articulating surface. This rollback is guided by the posterior cruciate ligament (PCL). The asymmetric rollback results in the tibia internally rotating relative to the femur during flexion. During flexion the lateral femoral condyle moves posteriorly while the position of the medial femoral condyle is relatively stationary. This produces relative internal tibial rotation or external femoral rotation during knee flexion. At high degrees of flexion the lateral femoral condyle may displace posteriorly to the point that it is partially subluxed from the tibial surface. The combined lateral femoral roll-back and femoral external rotation are necessary to permit large degrees of knee flexion.

In order to achieve the goal of relatively normal kinematic function after total knee arthroplasty, our tibial component configuration produces greater posterior translation of the lateral than the medial femoral condyle during knee flexion. Differential geometries of the medial and lateral tibial bearing surfaces provide selective guided posterior rollback on the lateral plateau. The tibial bearing geometry is designed as shown in FIG. 9 and FIG. 10 to achieve the desired natural knee kinematics.

Referring to FIG. 9, the medial articulating geometry 202 is dished or concave and the lateral articulating geometry 201 is posteriorly sloped and without a posterior lip. Here the angle α is the posterior slope of the medial articulating geometry 202 and the angle β is the posterior slope of the lateral articulating geometry 201. The posterior slope β of the lateral articulating geometry is greater than the posterior slope α of the medial articulating geometry. Optimally the posterior slope β on the lateral articulating surface is 1 to 2 degrees more than the posterior slope a on the medial articulating surface. The greater slope of the lateral articulating geometry allows the femoral component condyle to roll down the posterior during knee flexion. This tibial bearing component for a prosthetic knee implant system is particularly well-adapted for knee flexes beyond 130 degrees.

Referring to FIG. 11, this is a frontal view cross-section of the femoral component 100 and tibia insert component 200. Here R1 is the radius of the medial condyle and R2 is the radius of the lateral condyle of the femoral component 100 and R3 is the radius on the medial articulating geometry and R4 is the radius of the lateral articulating geometry of the tibial insert 200. In this invention the components are designed with a wide femoral condyle with the radii R1 and R2 matched to the corresponding tibia articulating surface radii R3 and R4. The match is suitably to a ratio of 1:1 to 1.09:1 and we have optimised at 1.07:1 in the frontal plane throughout the full range of motion, thereby maximizing contact area and reducing stress that can lead to premature polyethylene wear. This design satisfies conflicting needs of both resistance to wear and natural kinematics.

1. An artificial knee joint which comprises: a femoral component to be attached to a femur, the femoral component having a posteromedial condyle and a posterolateral condyle; and a tibial component to be attached to a tibia, the tibial component having a medial articulating geometry and a lateral articulating geometry, wherein in the femoral component the width and offset of the posteromedial condyle is greater than the width and offset of the posterolateral condyle and in the tibial component the angle of the posterior slope of the lateral articulating geometry is greater than the angle of the posterior slope of the medial articulating geometry.

2. An artificial knee joint as claimed in claim 1, wherein in the femoral component the offset of the posteromedial condyle is 1 to 3 mm greater than the offset of the posterolateral condyle.

3. An artificial knee joint as claimed in claim 2, wherein in the femoral component the offset of the posteromedial condyle is 2.5 mm greater than the offset of the posterolateral condyle.

4. An artificial knee joint as claimed in claim 1, wherein in the femoral component the width of the posteromedial condyle is 1 to 2 mm greater than the width of the posterolateral condyle.

5. An artificial knee joint as claimed in claim 4, wherein in the femoral component the width of the posteromedial condyle is 1.5 mm greater than the width of the posterolateral condyle.

6. An artificial knee joint as claimed in claim 1, wherein in the tibial component the angle of the posterior slope of the lateral articulating surface is 1 to 2 degrees greater than the angle of the posterior slope of the medial articulating surface.

7. An artificial knee joint as claimed in claim 1, wherein in the femoral component the posterior slope of the lateral articulating surface drops to a level at the rear edge of the tibial component that is 1 to 2 mm lower than the level that the posterior slope of the medial articulating geometry drops to at the rear edge of the tibial component.

8. An artificial knee joint as claimed in claim 1, wherein in the frontal plane the radius on the tibial component and the radius on the femoral component are in the ratio 1:1 to 1.09:1.

9. An artificial knee joint as claimed in claim 8, wherein in the frontal plane the radius on the tibial component and the radius on the femoral component are in the ratio 1.07:1.
